(19) United States
(12) Patent Application Publication
(10) Pub. No.: US 2007/0043311 A1
(43) Pub. Date: Feb. 22, 2007

(54) Sling assembly for supporting an arm of a user

(76) Inventor: Cintra Jaggan-Vince, London (GB)

Correspondence Address: Iandiorio & Teska, 260 Bear Hill Road, Watham, MA, 02451, US

(21) Appl. No.: 10/574,049

(22) PCT Filed: Sep. 21, 2004
(86) PCT No.: PCT/GB04/04011

§ 371 Date: Mar. 28, 2006

(30) Foreign Application Priority Data
Sep. 30, 2003 (GB) 0322909.3

(51) Int. Cl. A61F 5/00

(52) U.S. Cl. 602/4

(57) ABSTRACT

A sling assembly (60) for supporting an arm (62) of a user (64), which sling assembly (60) comprises a support portion for being worn by the user (66), a sling portion (68) for supporting the arm of the user, and fastener means (70) for fastening the sling portion (68) to the support portion (66), the fastener means (70) being a releasable fastener means (70) for enabling the sling portion (68) to be fastened and unfastened with respect to the support portion.

This invention relates to a sling assembly for supporting an arm of a user. The sling assembly is especially useful for persons recovering from a stroke or other conditions causing paralysis of an arm.

Known simple slings are made from a triangular bandage which is placed around a users arm and tied behind the user's neck. More complex known slings comprise a sling portion for supporting the arm of the user, and support straps which extend over the user's shoulder or around the users neck. Often the sling portion is made of a foam material.

The known slings have various disadvantages which make them unsuitable for a user having a paralysed arm. More specifically, many known slings are difficult to put on without help. This means that a user is unlikely repeatedly to be able to take the sling on and off throughout a day, unless there is someone there to help. A person with arm paralysis often does not need to wear a sling constantly, and may only need to support the paralysed arm periodically.

Many known slings are uncomfortable to wear. A sling which ties around the users neck, or which is worn over a users shoulder, can cause pain because the neck or shoulder is required to support the weight of the arm in the sling. In the case of a person with arm paralysis, the problem can be acute because the arm is not able to support itself and therefore the neck or shoulder of the user is supporting the entire weight of the arm. A sling which is uncomfortable to wear can affect a user's movement and posture, in addition to being uncomfortable.

In addition, many known slings are unattractive and not cosmetically pleasing to the user.

Known slings that comprise a foam support portion often create pressure on a small section of the user's arm or wrist, with this pressure having the potential to cause damage. For example, a user with a paralysed arm has little or no sensation in the arm and the user will not feel the pressure created by the sling. This may result in damage to the arm or wrist, because the user will not realise that the sling should be moved. Also, some known slings, especially those made from triangular bandages, elevate the arm. This constricts the elbow joint which can result in a reduced blood flow through the arm.

It is an aim of the present invention to obviate or reduce at least some of the above mentioned problems.

Accordingly, in one non-limiting embodiment of the present invention, there is provided a sling assembly for supporting an arm of a user, which sling assembly comprises a support portion for being worn by the user, a sling portion for supporting the arm of the user, and fastener means for fastening the sling portion to the support portion, the fastener means being a releasable fastener means for enabling the sling portion to be fastened and unfastened with respect to the support portion.

The sling assembly of the present invention is able to be put on and taken off more easily than many known slings. This is because the fastener means enables the sling portion to be separated from the support portion if required before putting the support portion on or taking the support portion off. In addition, the fastener means enables the sling portion to be removed from the support portion as and when desired so that the arm can be moved for exercise purposes whilst the arm is still in the sling portion. If desired, the arm can be removed from the sling portion for exercise or other purposes, with the support portion still being worn. Because the support portion is still being worm, the nuisance of having to put the support portion on and off each time it is desired to move the arm is avoided.

Thus the sling assembly of the present invention enables a user to go about daily life more easily and in greater comfort than with many known slings. The sling assembly may be produced to be cosmetically more appealing than known slings. The sling assembly is easier to use than many known slings so that, for example, the support portion may be put on, then the arm placed in the sling portion, and then the sling portion can be connected to the fastener portion using the fastener means. This procedure may be reversed if it is desired to remove the arm from the sling portion after a period of use.

Preferably, the sling assembly includes adjuster means for adjusting the sling portion with respect to the support portion and thereby to enable the arm of the user to be supported in an optimum comfortable position. The use of the adjuster means compares favourably with most known slings which are designed to hold an arm of a user in one position only. This is undesirable for many users, for example users with a paralysed arm. More specifically, a user with a paralysed arm which is held in one position only by a sling may suffer loss of mobility in joints in the arm, and especially at the elbow joint. Prolonged wear of the known slings may additionally result in strictures. With the use of the adjuster means, it is possible to adjust the position of the arm to find an optimum comfortable position at any one time. This in turn helps to avoid loss of mobility of joints and the onset of strictures.

The fastener means and the adjuster means may be integrally formed with each other. Alternatively, the fastener means and the adjuster means may be separately formed from each other.

The support portion may be a harness. The harness may have a ring portion for fitting over a head of the user and being worn around a neck of the user.

Alternatively, the support portion may be a jacket. The jacket may be a sleeveless jacket. Preferably the jacket has one sleeve only. In this case, the jacket may be worn over one shoulder of the user, and preferably also around a waist of a user. Such a jacket may be especially comfortable for the user because the weight of the supported arm in the sling portion is able to be spread across the back, shoulder and about the waist of the user.

If desired, the jacket may have two sleeves. In this case, the jacket may be worn over both shoulders of the user, and also around the waist of the user if desired. The wearing of the jacket over both shoulders will further spread the load of the arm in the sling portion.

The sling portion may be a folded sheet member. Thus, for example, the sling portion may be made up of two substantially identical halves on either side of a fold line. The two halves are preferably substantially trapezoidal in shape. In use, the arm in the sling portion will rest generally about the fold line, with the two halves positioned one on either side of the supported arm.

The sling assembly may be made from any suitable and appropriate materials. Thus, for example, the sling assembly may be made of waterproof material. This then allows the sling assembly to be worn when the user is washing, showering or bathing.

If desired, the sling assembly may be made from composite materials. Thus different layers of wadding may be employed, covered by an appropriate outer covering material. Wadding or other material of different degrees of softness may be employed at different places in the sling assembly. The support portion may be made from the same or different materials as the sling portion.

The material for the sling assembly, for example a fabric covering and/or a wadding or other filling, may advantageously be flame retardant for safety reasons.

Embodiments of the invention will now be described solely by way of example and with reference to the accompanying drawings in which:

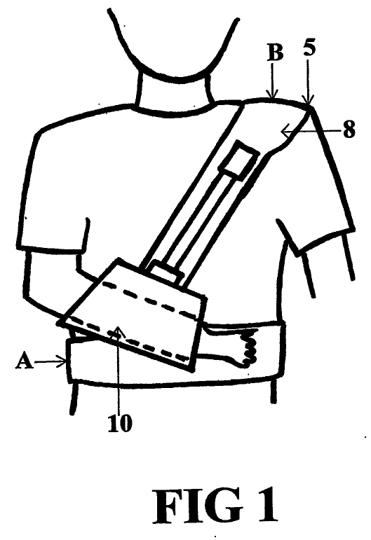

FIG. 1 shows a first sling assembly for supporting an arm of a user;

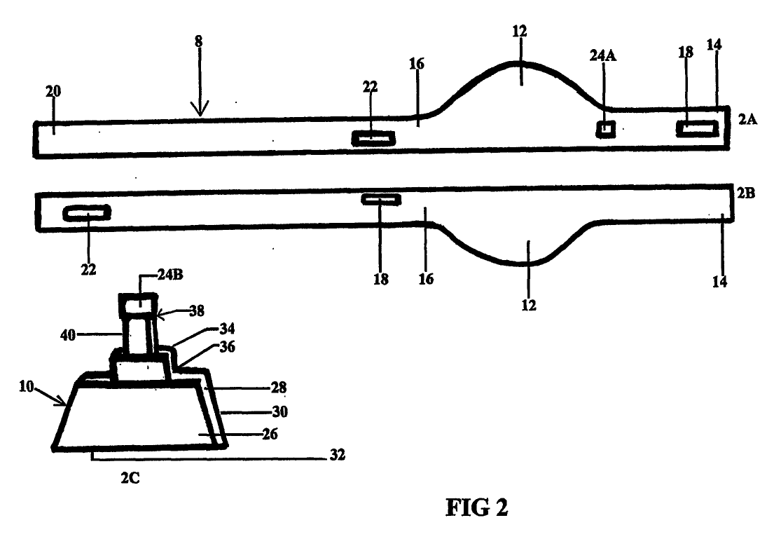

FIG. 2 is an exploded view of various parts of the sling assembly shown in FIG. 1;

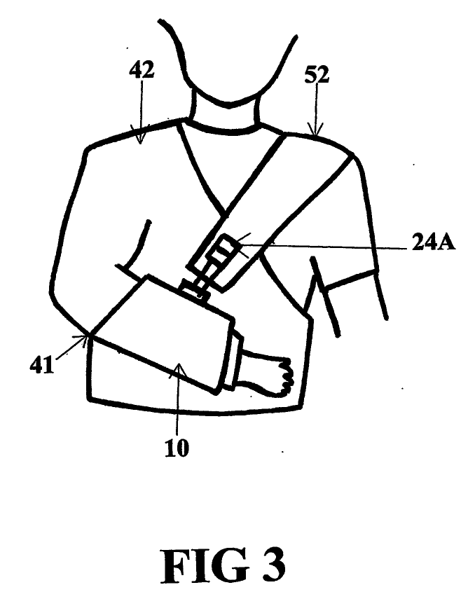

FIG. 3 shows a second sling assembly which is being worn and which is supporting an arm of a user.

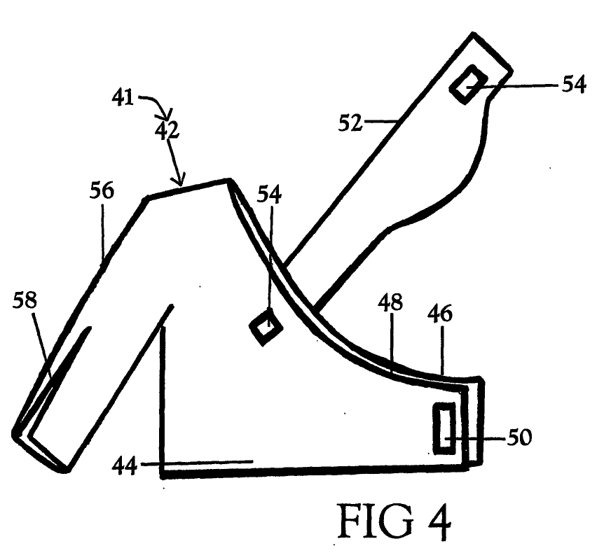

FIG. 4 shows the sling assembly of FIG. 3 not being worn;

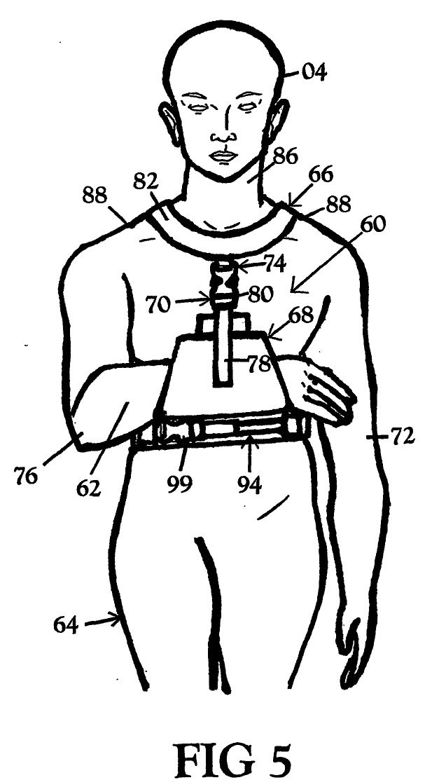

FIG. 5 shows a third sling assembly;

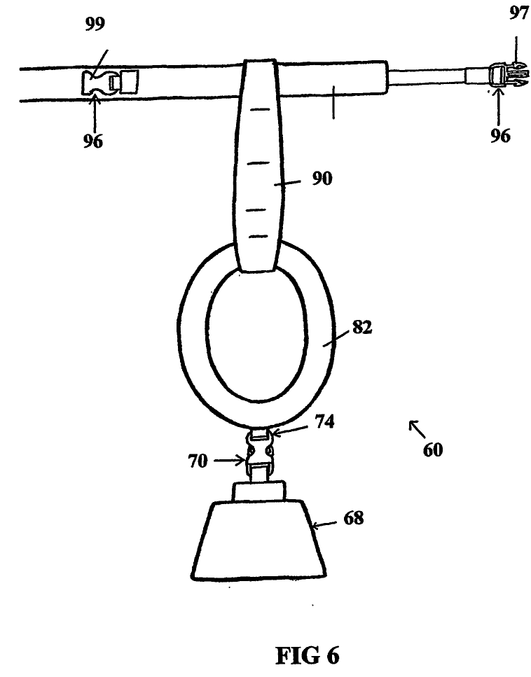

FIG. 6 shows the sling assembly of FIG. 5 not being worn;

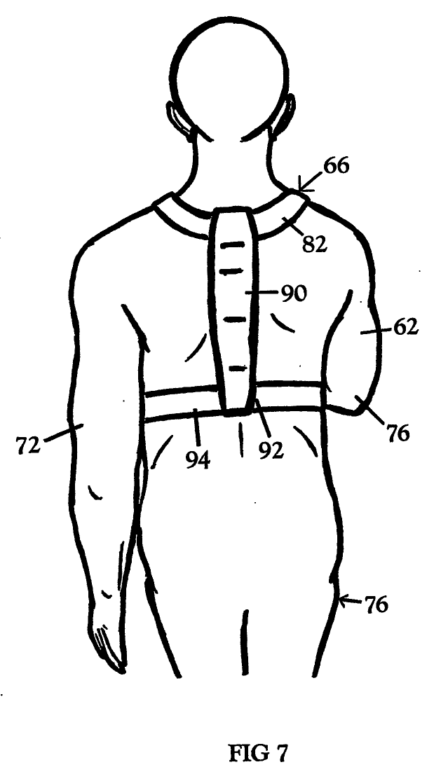

FIG. 7 is a rear view of the sling assembly of FIG. 5 as worn,

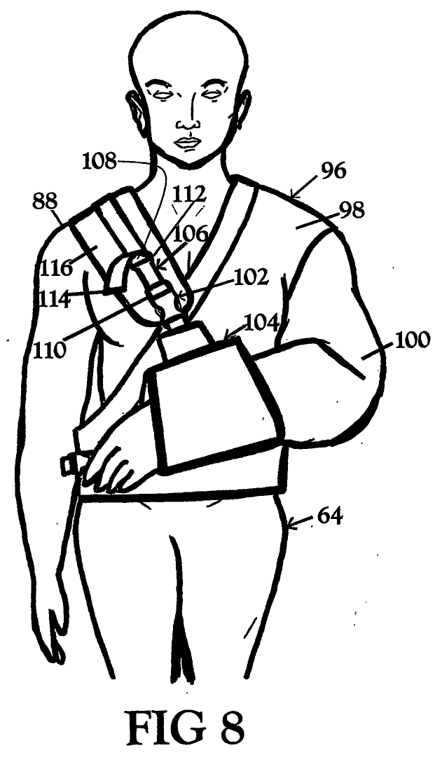

FIG. 8 shows a fourth sling assembly being worm;

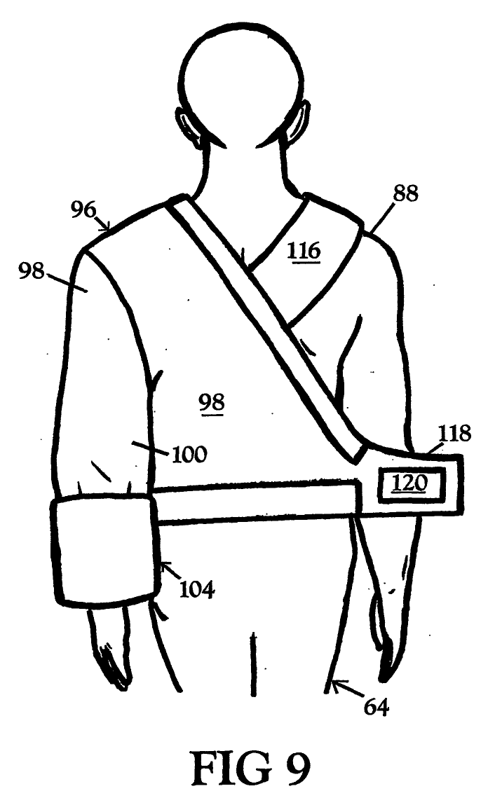

FIG. 9 is a rear view of the sling assembly of FIG. 8 as worn;

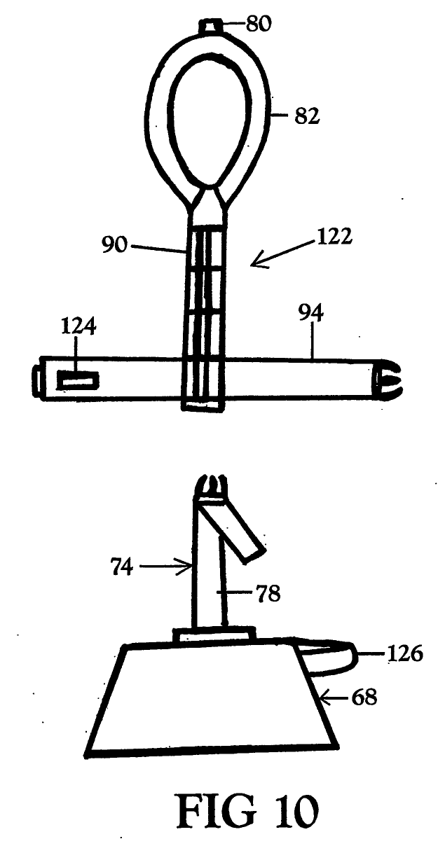

FIG. 10 shows a fifth sling assembly; and

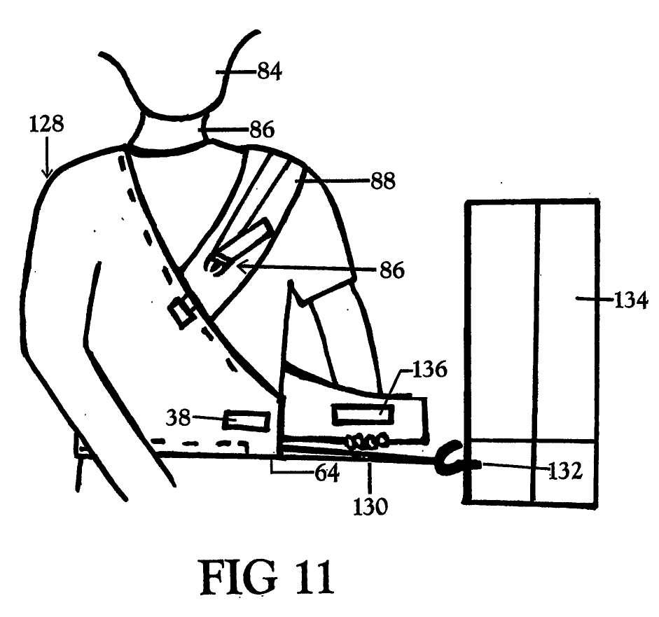

FIG. 11 shows a sixth sling assembly.

Referring to FIGS. 1 and 2, there is shown a sling assembly 5 for supporting an arm of a user. The sling assembly 5 comprises a support portion 8 for being worn by the user, and a sling portion 10 for supporting the arm of a user. The support portion 8 is constructed for being worn over the users shoulder and around the users waist as shown in FIG. 1.

Referring especially to FIG. 2, it will be seen that the support portion 8 is elongate in length and it includes a widened portion 12 for being worn over the user's shoulder. The support portion 8 is assembled by placing one end 14 at one side of the user's body at waist level, as indicated by arrow A in FIG. 1. The support portion 8 is then passed over the user's opposite shoulder, as shown by arrow B in FIG. 1. The support portion 8 is then passed across the users back so that the middle 16 of the support portion 8 meets the first end 14 at the position shown by arrow A. The first end 14 and the middle 16 are provided with complementary fastener means 80 such for example as Velcro (Registered trade mark) strips, press studs or the like. The fastener means 18 enable the first end 14 and the middle 16 to be joined together. The remainder of the support portion 8 is passed around the users waist, so that a second end 20 of the support portion 8 meets the middle 16 at the side of the users waist at the position shown by arrow A. The middle 16 and the second end 20 the support portion 8 are provided with further fastener means 22 so that the middle 16 and the second end 20 may be joined together. The fastener means 22 may be the same or different from the fastener means 18.

The sling assembly is provided with a first part 24A of fastening means 24 for connecting together the support portion 8 and the sling portion 10. More specifically, the fastener means 24 comprises the first part 24A which is provided on the support portion 8, and also a second part 24B which is provided on the sling portion 10. The first and second parts 24A, 24B are able to be fastened together to enable the sling portion 10 to be attached to the support portion 8, and they are able to be unfastened in order to enable the sling portion 10 to be removed from the support portion 8.

The sling portion 10 comprises a body part 26 for supporting the arm. The body part 26 is padded to cushion the arm. The body part 26 comprises two halves 28, 30 on either side of a fold line 32.

In use of the sling assembly, the arm rests on the fold line 32. The two halves 28, 30 are generally trapezoidal in shape. Each half 30, 32 has a joining portion 34, 36 on an edge opposition to the fold line 32. Each joining portion 34, 36 is attached to a strap 38, 40. The straps 38, 40 are attached to the second part 24B of the fastening means. In use, the first and second parts 24A, 24B of the fastening means are joined to attach the sling portion 10 to the support portion 8.

In an alternative embodiment of the sling assembly shown in FIGS. 1 and 2, a further strap may be added to the support portion 8 for being worn over the other shoulder of the user.

Referring now to FIGS. 3 and 4, there is shown a second sling assembly 41 having a support portion in the form of a jacket 42. The jacket 42 has a body portion 44 which comprises a back portion 46 and a front portion 48. The back and front portions 46, 48 are provided with fasteners at one side 50 of the user's waist in order to allow them to be joined together. A strap 52 is provided which is attached to the back portion 46, and passes over a shoulder of the user to meet the front portion 48, where further fasteners 54 are provided. The jacket 42 also has a sleeve 56 which covers the arm in the sling portion. The sleeve 56 is provided with a slit 58, in order to allow access to the arm in the sling portion. The jacket 42 is used in conjunction with the sling portion 10 shown in FIGS. 1 and 2. The jacket 42 is provided with the first part 24A of the fastening means 24 on the strap 52.

Referring now to FIGS. 5-7, there is shown a third sling assembly 60 for supporting an arm 62 of a user 64. The sling assembly 60 comprises a support portion 66 for being worn by the user 64, a sling portion 68 for supporting the arm 62 of the user 64, and fastener means 70 for fastening the sling portion 68 to the support portion 66. The fastener means 70 is a releasable fastener means 70 for enabling the sling portion 68 to be fastened and unfastened with respect to the support portion 66.

The sling assembly 60 is easy to be put on because the support portion 66 can first be put on prior to connecting the sling portion 68 to the support portion 66 using the fastener means 70. In addition, because the sling portion 68 can be removed from the support portion 66, the arm 62 can be moved for exercise purposes whilst the arm 62 is in the sling portion 68 or, alternatively, whilst the arm 62 has been removed from the sling portion 68. The movement of the arm 62 if is it paralysed can be effected by the other arm 72 of the user 64 or alternatively, the movement of the arm 62 can be effected by another person (not shown) helping the user 64.

The sling assembly 60 includes adjuster means 74 for adjusting the sling portion 68 with respect to the support portion 66. This enables the arm 62 of the user 64 to be supported in an optimum comfortable position. This in turn helps to avoid the arm becoming set in one single position and thereby causing loss of mobility at the elbow joint 76 and also possibly causing strictures in the arm 62. The adjuster means 74 can be a pull strap passing through a buckle so that the adjuster means may then be like the adjuster means employed for aircraft seat belts. The fastener means 70 may also be like the fastener means employed on aircraft seat belts.

The adjuster means 74 is formed as an integral part of the fastener means 70. More specifically, the fastener means 70 comprises a strap 78 which passes through a loop 80 on the support portion 66. By pulling on the strap 78, the sling portion 68 can be raised or lowered with respect to the support portion 66. Thus the sling portion 68 is able to be adjusted by the strap 78, in addition to being connected to the support portion 66 by the strap 78. When the sling portion 68 is at its desired position, then the strap 78 can be secured in position by locking means as employed in aircraft seat belts, or the strap may be attached to itself by fastener means such for example as Velcro strips, press studs or other suitable and appropriate fastener means.

As can be seen from FIGS. 5-7 the support portion 66 is in the form of a harness. The harness has a ring portion 82 which fits over the head 84 of the user 64 and fits around their neck 86, resting on the user's shoulders 88. The ring portion 82 is connected to an elongate portion 90 which extends down the user's back as shown in FIG. 7. The elongate portion 90 has a loop 92. A waist strap 94 passes through the loop 92. If desired, several loops 92 may be provided vertically above each other so that the waist strap 94 can pass through a loop 92 that puts the waist strap 94 at an appropriate height. Such an adjustment facility enables the support portion 66 to be adjustable and comfortably worn by persons of different sizes. The waist strap 94 is secured to itself by means of a buckle. The buckle 96 has a first locking part 97 which pushes into and locks in a second housing part 99. Adjustment can be effected by pulling on a free end of the waist strap 94. Thus, the waist strap 94 may be constructed like an aircraft seat belt.

Referring to FIGS. 8 and 9, there is shown a fourth sling assembly 96 being worn by a user 64. The sling assembly 96 is such that it has a support portion 98 in the form of half a jacket with one sleeve 100. Fastener means 102 enables a sling portion 104 to be connected to the support portion 98. Adjuster means 106 is provided for adjusting the height of the sling portion 104 with respect to the support portion 98. The adjuster means 106 extends over a shoulder 88 of the user 64 as shown. The adjuster means 106 comprises a strap 108 which passes through fastener devices, 110, 112. Adjustment to raise the sling portion 104 is effected by pulling on a free end 114 of the strap 108. Lowering of the sling portion 104 is effected by loosening the strap 108 in the fastener devices 110, 112. The fastener means 102 is supported on a band member 116 which is wide than the strap 108 and which ensures that the strap 108 does not cut into the shoulder 88 during use of the sling assembly 96.

FIG. 9 shows how the support portion 118 has a waist part 110 which is provided with a Velcro strip 120. The waist part 118 wraps around the remainder of the support portion 98 and the Velcro strip 120 attaches to another Velcro strip (not shown).

Referring now to FIG. 10, there is shown a fifth sling assembly 122 which is like the third sling assembly 60. Similar parts in both Figures have been given the same reference numerals for ease of comparison and understanding. In FIG. 10, fastening of the strap 94 is effected by Velcro pads 124 instead of the buckle 96. Also, in FIG. 10, the sling portion 68 is provided with a loop 126 which, during use of the sling assembly 122 enables the upper arm and shoulder to be held firmly.

Referring now to FIG. 11, there is shown a sixth sling assembly which is like the sling assembly 41 shown in FIGS. 3 and 4. Similar parts have been given the same reference numerals for ease of comparison and understanding. In FIG. 11, it will be seen that the sling assembly 128 is provided with a loop 130 which fits to a hook 132 on a door 134. The use of the loop 130 and the hook 132 enables the user 64 on their own and without the aid of a helper to get the waist portion of the sling assembly tightly around their waist so that then the Velcro pads 136, 138 can be placed together and thereby secure both ends of the waist portion of the support portion around the user 64.

It is to be appreciated that the embodiments of the invention described above with reference to the accompanying drawings have been given by way of example only and that modifications may be effected. Thus, for example, the illustrated support portions and sling portions may be of a different shape to those shown. Also, other types of fastener means and adjuster means may be employed. The fastener means and the adjuster means may be separate from each other or integral with each other as may be desired.

1. A sling assembly for supporting an arm of a user, which sling assembly comprises a support portion for being worn by the user, a sling portion for supporting the arm of the user, and fastener means for fastening the sling portion to the support portion, the fastener means being a releasable fastener means for enabling the sling portion to be fastened and unfastened with respect to the support portion.

2. A sling assembly according to claim 1 and including adjuster means for adjusting the sling portion with respect to the support portion and thereby to enable the arm of the user to be supported in an optimum comfortable position.

3. A sling assembly according to claim 1 in which the fastener means and the adjuster means are integrally formed with each other.

4. A sling assembly according to claim 1 in which the fastener means and the adjuster means are separately formed from each other.

5. A sling assembly according to claim 1 in which the support portion is a harness.

6. A sling assembly according to claim 5 in which the harness has a ring portion for fitting over a head of the user and being worn around a neck of the user.

7. A sling assembly according to claim 1 in which the support portion is a jacket.

8. A sling assembly according to claim 7 in which the jacket has one sleeve only.

9. A sling assembly according to claim 1 in which the sling portion is a folded sheet member.

10. A sling assembly according to claim 1 in which the fastener means comprises a plurality of interlocking plastics members which press together and pull apart.

11. A sling assembly according to claim 1 in which the fastener means comprises at least one press stud.
